(19) United States
(12) Patent Application Publication
(10) Pub. No.: US 2005/0004562 A1
(43) Pub. Date: Jan. 6, 2005

(54) Method and equipment for controlling necrotized areas in ablative technique on tissues

(76) Inventors: Luciano Alcidi, Sesto Fiorentino (IT); Gino Grassi, Sesto Fiorentino (IT)

Correspondence Address: NIXON & VANDERHYE, PC, 1100 N GLEBE ROAD, 8TH FLOOR, ARLINGTON, VA, 22201-4714, US

(21) Appl. No.: 10/490,586

(22) PCT Filed: Sep. 23, 2002
(86) PCT No.: PCT/IT02/00604

(30) Foreign Application Priority Data
Sep. 28, 2001 (IT) FI01A000176

(51) Int. Cl. A61B 18/18

(52) U.S. Cl. 606/34; 606/41

(57) ABSTRACT

The invention relates a method and equipment for controlling necrotized areas during operations using the ablative technique on tissues, where a volume of tissue is ablated by means of thermal action using an electrocatheter for ablation. Method and equipment are provided for measurement of an electrical parameter based on the hydration of the said tissue using at least one sensor located in correspondence with a peripheral zone of the tissue volume to be treated.

### FIELD OF THE INVENTION

This invention concerns a method and the equipment for controlling necrotized areas during operations effected by using ablative technique on tissues.

### PRIOR ART

There are many and various branches of surgery in which the endermic ablation method has been developed and used where the normal surgical technique would be too invasive or dangerous for the patient. This in an attempt to destroy the pathological tissues or those which are responsible for the pathologies in question.

Ablation is obtained in most cases by means of thermal action, whether by heating (hyperthermia) or by cooling (cryoablation), using an electrocatheter placed in the area to be treated.

In order to verify the correctness of the operation, these methods normally try to control the operational results by measuring the temperatures reached in the tissue to be ablated with special probes that are provided on the electrocatheter. However, the simple temperature value measured around the active electrode does not make it possible to know the volume of tissue which is effectively necrotized.

Many ablation devices keep the impedance measurement under control by means of a reading between the active electrode and the reference electrode, which usually consists of a reference plaque placed in contact with the patient's back. However, the measurement only serves to confirm that the interface (active electrode—tissue—reference plaque) maintains the same electrical characteristics required for a good ablative technique, but it cannot be used as an objective indicator of the correctness of the tissue ablation process. In fact, a sudden increase in the tissue impedance indicates both that the tissue around the active electrode is evaporating and/or being carbonized. In this case, the RF energy, namely the energy transmission means used by the ablators, is compromised.

### OBJECT AND SUMMARY OF THE INVENTION

The main object of this invention is to provide a method and the appropriate measuring equipment to give an effectively reliable evaluation of the state of the tissue to be ablated in real time and an indication that the necrotization of the area in question has been carried out. This object has been achieved, in accordance with the invention, by adopting the idea of creating a method and equipment with the characteristics described in the independent claims. Other characteristics of the invention are the subject of the dependent claims.

Among the advantages resulting from this invention there is the fact that the equipment and the method are extremely effective and capable of giving a precise real-time evaluation of the dimensions of the lesion (understood as the tissue necrotized by the treatment) produced during a Radio Frequency ablation; that they are relatively easy to make and operate; that the equipment requires extremely limited maintenance since its characteristics remain largely unaltered over time.

### BRIEF DESCRIPTIONS OF THE INVENTION

These and other advantages and characteristics of the invention will become clearer and more easily understood by technicians in this field from the description that follows and with the help of the enclosed drawings, which are included as practical examples of the invention but are not intended as limiting in any way, in which:

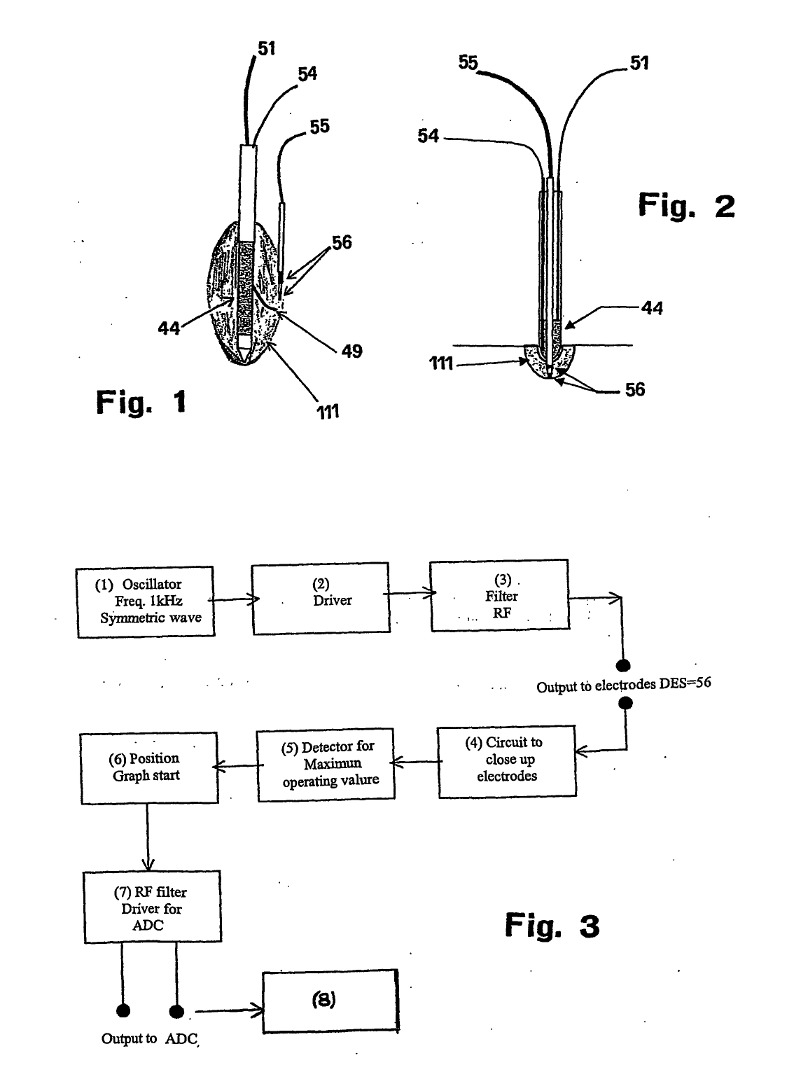

FIG. 1 shows a diagram of one possible version of the invention, in the treatment of parenchymal tissue, with separate measurement and ablation electrodes;

FIG. 2 shows a diagram of one possible version of the invention, in the treatment of heart muscle tissue, with measurement and ablation electrodes set along the same axis;

FIG. 3 shows a possible version of a circuit that forms part of the equipment included in the invention;

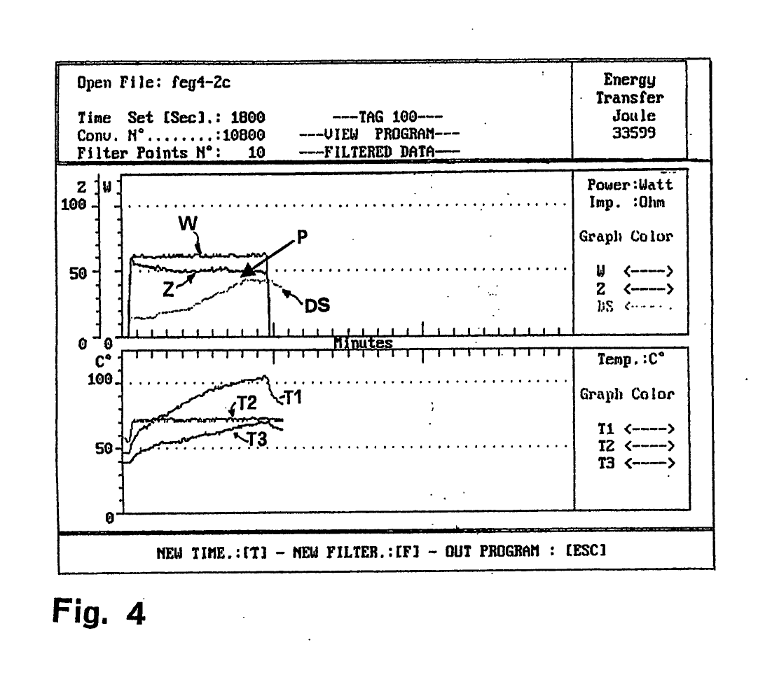

FIG. 4 shows a diagram of the results of a check test.

### DETAILED DESCRIPTION OF THE INVENTION

In accordance with the present invention, the method and equipment for the control of necrotized areas are based on the objective evaluation of the local dehydration process that manifests itself during the ablation process, by means of measuring the variations in local conductivity. This evaluation is made by observing the performance of the percentage value of a parameter called “Sectorial Electrodynamic Density” (SED), which is measured locally using a pair of electrodes inserted into the district concerned. This parameter is a parameter associated with the tissue hydration and is, in practice, an electrical parameter correlated with the conductivity presented by the tissue. Similarly, said parameter may be correlated with the impedance or the electric charges present (measured, by example, by means of pH), or with other parameters.

The term “district concerned” refers to the zone which is directly subjected to the ablation treatment, and which has been represented by on oval FIG. 111 on a darker background in FIGS. 1 and 2.

FIGS. 1 and 2 represent two possible versions of the equipment, in accordance with the present invention.

FIG. 1 shows an electrocatheter 44 for ablation, positioned within a district 111 that is to undergo treatment, and the number 51 indicates the connection to a source that supplies a radio frequency signal. The electrocatheter 44 is of the type furnished with one or more probes 49 for measuring the temperature, connected across the respective conductor 54. A device according to the present invention includes a pair of electrodes 56, which are designed to be placed in correspondence with the district concerned, in particular, in correspondence with a peripheral or border zone of the said district. The electrodes 56 are connected to a relative measuring circuit by means of the connection referred to with number 55 in the figures.

FIG. 2 also represents with number 54 the connection for the temperature measurement signal, while 51 is the connection to the RF source and 55 is the connection to the measurement circuit of the SED. In this case, too, the measurement electrodes 56 are placed in correspondence with the edge of the zone 111 to be treated.

One possible version of the measuring circuit is indicated in FIG. 3. The electrodes used for measuring the SED are set up, ideally, with a coaxial and dipolar structure, as in FIG. 1 and FIG. 2. This structure makes it possible to obtain an optimal resolution of the tissue conditions in which it is immersed. Furthermore, the dipole dimensions also determine the optimisation of the resolution. Small dipolar dimensions (e.g. 1-2 mm) are preferable for localising the SED variation with precision.

Tests carried out showed that it is possible to use monopolar sensors, with reference electrodes coinciding with the active ablation electrode. In this case, there will be lower resolution and more active filters will be needed for the RF. The advantage of using monopolar filters could lie in the fact that the electrodes are easier to create. In this case, too, the size of the measuring electrode plays an important role in localising the edge of the lesion.

The version with the monopolar sensor is not illustrated in the drawings, but FIG. 2 can be taken into consideration, hypothesising a single measuring electrode 56, with the use of electrode 44 as the reference electrode.

The block drawing in FIG. 3 may consist of the following:
Block (1) is represented by a free oscillator with a frequency of symmetric oscillation of 1 kHz; in the experiments carried out, an oscillator was used with a triangular wave of 5V amplitude.
Block (2) consists mainly of a current amplifier, which guarantees a good coupling with the sensor electrodes (56).
Block (3) is a filter for the radio frequency, guaranteeing good protection for the SED circuit.
Block (4) is a circuit of passive components that serves to close up the electrode ring and to register the useful signal.
Block (5) is a circuit consisting of an active detector of the maximum operating value at 1 kHz, registered by block (4).
Block (6) makes it possible to set the start value properly on the graph.
Block (7) represents the output circuit for the acquisition of data. Furthermore, it helps minimise the noise caused by the RF ablation.
Block (8), which is set downstream from the respective ADC device, represents the elaboration means used for the signal received. These means may consist of a personal computer and the relative software.

One possible method for actuating the present invention could make use of the instrumentation described below, and which was used in the testing carried out.

For the ablation generator (connected to connection 51 in FIGS. 1 and 2) use was made of a TAG-100 generator, which is available on the market and is made by Fogazzi S.n.c..

The catheter may be connected to a TAP-45 pump for electrode cooling, available on the market and made by Fogazzi S.n.c..

For the electrocatheter for ablation a catheter model MIRAS 40-30-360 with three thermocouples was used, available on the market.

The electrocatheter 56 for measuring the SED, as well as the instrumentation for measuring the SED, the ADC devices, the SED measurement circuits and the specific software are all experimental.

A portable personal computer with a 486 microprocessor was used for elaborating the data. Here below there follows a description of how the present invention can be applied.

It is already well-known how the cells forming any tissue are immersed in the interstitial liquid, consisting prevalently of water and molecules of various elements, such as sodium, potassium, etc. Some of these elements are ions and, together with the electrons, they contribute to defining the electrical conductivity of the environment. However, unlike the electrons, the molecular ions have a great mass and, therefore, their mobility depends upon their environment. During the ablation procedure, two distinct processes take place: there is a significant increase in the district temperature and the steady dehydration of the zone. From an electrical point of view, these two processes produce contrasting effects. In fact, while the rise in temperature increases the molecular thermal agitation, an excessive dehydration tends to impede this molecular agitation. The result of this is that during ablation the so-called Sectorial Electrodynamic Density (SED) at the start, in an environment which is hydrated normally, will have a value that registers a steady growth, due to the temperature rise, which is followed by a fall during the process of dehydration. Experiments carried out in vitro have shown that irreversible biological damage is caused when a plateau value is reached in the measurement zone (namely, the zone in which the measuring electrodes 56 are positioned), the start of this plateau value being indicated by the arrow P in the diagram of FIG. 4.

The upper box in FIG. 4 shows the behaviour of:
W) Power supply during the period;
Z) Ablation impedance (the impedance seen between the active electrode and the reference plaque);
DS) Performance of the Sectorial Electrodynamic Density.

The lower box shows the behaviour of the temperatures T1, T2 and T3, that is, the signals originating from the corresponding thermocouples presented by the electrode (as already mentioned, MIRAS—ioc 40-30-360). The sensors T1 and T2 are located on the body of the electrode, like that indicated with the number 49 in FIG. 1. During the testing, a pump of the TAP 45 type was used to cool the source electrode. As shown in the lower box of the diagram, the temperature measured by the thermocouple T1 rises freely (following the ablation treatment), while the temperature measured by the thermocouples T2 and T3 is automatically kept below 75° by the circulation of the cooling liquid.

Referring to the example in FIG. 7, the electrode for measuring the sectorial electrodynamic density was positioned at 2 cm from the source electrode. The ablation was interrupted as soon as the plateau was reached. Once a section of the tissue was taken, the evidence showed that the measuring electrode was situated at the edge of the lesion.

In practice, the details of the operation may vary in form, size, layout of the elements, the nature of the materials used, without leaving the confines of the invention and, consequently, they are still covered by the terms of the patent.

1-15. (canceled).

16. Equipment for controlling necrotized areas during operations using an ablative technique on tissues, where a volume of tissue is ablated by acting upon the volume of tissue by means of a thermal action using an electrocatheter for ablation, characterised by the fact of consisting of one or more sensors, designed in such a way as to be suitable for placing in correspondence with the peripheral zone of said volume of tissue and connected to a relative measuring circuit, which is provided with means for emitting and receiving and elaborating a signal that varies according to the hydration detected in the tissue.

17. Equipment according to claim 16, wherein said one or more sensors consist of a pair of electrodes connected to said means of emission and to said means of reception and elaboration.

18. Equipment according to claim 17, wherein one of said electrodes consists of an electrode of the electrocatheter of ablation.

19. Equipment according to claim 16, wherein said means for emitting a signal consist of a free oscillator, a current amplifier and a filter.

20. Equipment according to claim 16, wherein said means for receiving a signal consist of a circuit of passive components, an active measuring circuit, a positioning device, an outlet circuit that is connected to means for elaborating the signal.

21. Equipment according to claim 16, wherein said means for elaborating a signal consist of an ADC device and a personal computer provided with the relative software.

22. Equipment according to claim 16, wherein said signal is correlated to the electrical conductivity at that moment present in the tissue and derives from the elaboration of a signal obtained by the passage across a pair of electrodes, placed in said volume of tissue, of a pre-set signal emitted by a relative circuit.

23. Equipment according to claim 16, wherein said signal is correlated to the impedance at that moment presented by the tissue and derives from the elaboration of a signal obtained from the passage across a pair of electrodes, placed in said volume of tissue, of a pre-set signal emitted by a relative circuit.

24. Equipment according to claim 16, wherein said signal is correlated to the electrical charges at that moment presented by the tissue and derives from the elaboration of a signal obtained by the passage across a pair of electrodes, placed in said volume of tissue, of a pre-set signal emitted by a relative circuit.

25. Method for controlling necrotized areas during operations using the ablative technique on tissues, where a volume of tissue is ablated by means of thermal action using an electrocatheter for ablation, comprising the step of measurement of an electrical parameter based on the hydration of the said tissue using at least one sensor located in correspondence with a peripheral zone of the tissue volume to be treated.

26. Method according to claim 25, characterised in that said electrical parameter is correlated to the electric conductivity that is revealed by the tissue at that instant, and derives from the elaboration of a signal obtained from the passage across a pair of electrodes, located in said volume of tissue, of a pre-set signal emitted by a relative circuit.

27. Method according to claim 25, characterised in that said electric parameter is correlated to the inductance revealed by the tissue at that instant and derives from the elaboration of a signal obtained from the passage across a pair of electrodes, located in said volume of tissue, of a pre-set signal emitted by a relative circuit.

28. Method according to claim 25, characterised in that said electric parameter is correlated to the electrical charges at that moment present in the tissue and derives from the elaboration of a signal obtained from the passage across a pair of electrodes, located in said volume of tissue, of a pre-set signal emitted by relative circuit.

29. Method according to claim 25, characterised by the fact of setting up, in correspondence with said volume of tissue, at least one measuring electrode, connected to a relative measuring circuit and a reference electrode presented by said ablation catheter.

30. Method according to claim 25, characterised by the fact of placing a pair of measuring electrodes in correspondence with said volume of tissue, said measuring electrodes being connected to a relative measuring circuit.
